(19) United States
(12) Patent Application Publication
(10) Pub. No.: US 2008/0317728 A1
(43) Pub. Date: Dec. 25, 2008

(54) Lowering Uric Acid to Prevent or Accelerate Recovery of Acute Renal Failure

(75) Inventor: Richard J. Johnson, Gainesville, FL (US)

Correspondence Address: Beusse Wolter Sanks Mora & Maire, 390 N. ORANGE AVENUE, SUITE 2500, ORLANDO, FL, 32801, US

(73) Assignee: UNIVERSITY OF FLORIDA RESEARCH FOUNDATION, INC., Gainsville, FL (US)

(21) Appl. No.: 11/570,902

(22) PCT Filed: Jun. 21, 2005
(86) PCT No.: PCT/US2005/021771

§ 371 Date: Dec. 19, 2006

Related U.S. Application Data
(60) Provisional application No. 60/581,687, filed on Jun. 21, 2004.

(51) Int. Cl. A61K 38/44; A61P 13/12

(52) U.S. Cl. 424/94.4

(57) ABSTRACT

Disclosed herein are methods of preventing acute renal failure, ARF, as well as treating and accelerating the recovery of patients experiencing ARF. The inventors believe that an elevated serum uric acid, either occurring at baseline in the patient, or being brought about post-operatively is a major factor leading to ARF. Specifically exemplified are methods that involve administering to a patient at risk of experiencing ARF a composition comprising a uric acid lowering agent.

### BACKGROUND OF THE INVENTION

Over 700,000 cardiothoracic surgeries are performed annually in the United States (www.americanheart.org). The overall postoperative mortality rate for cardiovascular surgery is 2.2%. Thakar C V, Liangos O, Yared J P, Nelson D, Piedmonte M R, Hariachar S, Paganini E P. ARF after open-heart surgery: influence of gender and race. Am J Kidney Dis 2003; 41(4):742-751. The major risk factor for cardiovascular mortality is post-operative ARF requiring dialysis. Abel R M, Buckley M J, Austen W G, Barnett G O, Beck C H, Fischer J E. Etiology, incidence, and prognosis of renal failure following cardiac operations. J Thorac Cardiovasc Surg 1976; 71(3):323-333. When cardiovascular surgery is complicated by acute renal failure, the mortality rate worsens from 2.2% to 14% (defined as a doubling of creatinine) to over 60% (those requiring dialysis) Thakar et al.; and Chertow G M, Lazarus J M, Christiansen C L, Cook E F, Hammermeister K E, Grover F, Daley J. Preoperative risk stratification. Circulation 1997; 95:878-884. Thus, identifying ways to prevent acute renal failure may have major impact on the outcome of cardiovascular surgery, and other surgical procedures.

The most important predisposing factors for acute renal failure are impaired preoperative renal function (creatinine >1.5 mg/dL), decreased cardiac function (LVEF <40%, NYHA Class III/IV) and type of surgery. The key role for preoperative renal function was reported by Thakur in 22,589 subjects undergoing CV surgery (1). In this group, a serum creatinine >1.7 mg/dl was associated with postoperative ARF (doubling of creatinine) in 9% with dialysis in 4.3%; this increased to 19% and 4.9%, respectively, if the serum creatinine was >2.0 mg/dl (>180 mmol). Thakar et al. Leacche reported that in the absence of preexisting renal insufficiency the preoperative factors most commonly present in subjects who developed postoperative ARF was underlying cardiac function, with 36/40 subjects having preoperative NYHA class III or IV heart failure Leacche M, Rawn J D, Mihaljevic T, Lin J, Karavas A N, Paul S, Bryne J G. Outcomes in patients with normal serum creatinine and with artificial renal support for acute renal failure developing after coronary artery bypass grafting. Am J Cardiol 2004; 93(3):353-356. In these patients with poor cardiac function (LVEF <35%), despite the presence of normal renal function, the operative mortality exceeded 72%, with an overall 1-year survival of 10%, and a requirement for permanent dialysis in 64% of the survivors. Leachhe et al.

At the University of Florida approximately 500 CV surgeries are performed per year (defined as bypass, valve and thoracic aortic aneurysm repair). In this tertiary hospital which takes high risk subjects, approximately 15% of patients have a baseline creatinine of >1.5 mg/dl and 6.5% have both an elevated creatinine and LV ejection fraction of <40%. In a preliminary review of past data, the inventors have found that the presence of renal insufficiency (defined as serum creatinine of >1.5 mg/dl) is associated with a risk for postoperative dialysis-dependent renal failure of approximately 10% independent of cardiac status, and that for subjects with preserved renal function but impaired CV function (EF <40%) the frequency is 5%. This contrasts with an incidence of 0.5% in subjects with normal renal and cardiac function.

While it is difficult to extrapolate the results of our tertiary hospital to nationwide statistics, the data suggest the possibility that a significant number of subjects (up to 15-20% of patients undergoing CV surgery) may carry an increased risk for ARF deserving some type of prophylactic therapy. It is for this reason that identifying strategies to prevent ARF is our paramount importance in this population.

### SUMMARY OF THE INVENTION

According to certain embodiments, the subject invention pertains to methods for preventing, and/or accelerating recovery of ARF. In a specific embodiment, the subject invention pertains to methods of preventing and/or accelerating recovery of ARF occurring post-operatively. According to one aspect, a uric acid lowering agent (UALA) is administered to a patient immediately before, during or immediately after an operative procedure on the patient. In another aspect, the serum uric acid levels are monitored following administration of the UALA.

### BRIEF DESCRIPTION OF THE DRAWINGS

FIG. 1 is a graph demonstrating the rise in serum uric acid levels that occurs following cardiovascular surgery.

FIG. 2 Effect of afferent arteriole thickening induced by hyperuricemia on hemodynamic parameters in normal rats. Values of serum uric acid positive correlated with AA media/lumen (a). Arteriolopathy was associated with glomerular pressure (b). Thickening of AA negatively correlated with SNGFR (c) and positively correlated with afferent resistance (d). ◯ Control; Oxonic acid; OA+ Allopurinol.

FIG. 3 Effect of afferent arteriole thickening induced by hyperuricemia in RK rats. Values of serum uric acid positive correlated with AA media/lumen (a). Arteriolopathy was associated with glomerular pressure (b). Tubulointerstitial infiltration of CD5+ cells (lymphocytes) correlated with AA thickening (c). Glomerular pressure correlated with túbulointerstitial fibrosis (d). □ Control RK; ▪ Oxonic acid; ▪ OA+Allopurinol.

FIG. 4 is a graph showing the relationship of serum uric acid and serum nitrites at 1 and 7 Days of hyperuricemic induced rats. Serum was analyzed for uric acid concentration and nitrites/nitrates (NOₓ) by chemiluminescence method.

FIG. 5 represents a graph that shows the linear correlation of serum uric acid and serum nitrites.

### DETAILED DESCRIPTION OF THE INVENTION

The pharmaceutical compositions provided herein contain therapeutically effective amounts of one or more agents to lower uric acid that are useful in the treatment or amelioration of an elevated serum uric acid associated with certain operative procedures. The inventors have discovered that reducing uric acid levels associated with such procedures substantially decreases the chances of post-operative acute renal failure, and/or accelerates recovery time if post-operative acute renal failure occurs.

Furthermore, certain invention method embodiments are useful for prevention of or deceleration of progressive renal failure for circumstances not involving elevated uric acid levels brought about by surgical stress. The inventors have made the remarkable discovery that chronic elevated serum uric acid levels are not just associated with certain types of renal failure, but are indeed are primary cause or inducer of progressive renal failure.

The term “acute renal failure” or ARF as used herein refers to elevated creatinine serum levels of at least 1.5 times higher than baseline levels. In most patients. ARF is defined as serum creatinine levels 2 times normal levels. An elevated uric acid is defined as a serum uric acid >5.5 mg/dl.

The term “uric acid lower agent” or UALA refers to substances known to lower serum uric acid levels in mammals. UALAs include, but are not limited to, xanthine oxidase inhibitors such as allopurinol, hydroxyakalone, TEI-6720, febuxostat, and y-700; uricosurics such as benziodarone, benzbromarone, probenecid; uricase derivatives such as Rasburicase and Pegylated uricase; and gene based therapies such as uricase overexpression or blockade of URAT-1.

The methods taught herein are particularly useful against ARF brought about post-operative increases in uric acid. Accordingly, the subject methods may be used as a treatment following numerous surgical procedures, wherein such procedures are likely to lead to post-operative elevation in serum uric acid including, but not limited to, cardiovascular surgery; prolonged orthopedic surgeries; organ transplantation; abdominal/GI-related surgery; gynecological-related surgery, etc. In the most typical scenario, the subject methods are used in conjunction with cardiovascular surgeries. However, this method may be applied to the prophylaxis of any procedure in which an increased risk for acute renal failure is predicted (This would include the administration of contrast agents, nephrotoxins, or other types of surgeries).

The compounds are preferably formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives is (are) mixed with a suitable pharmaceutical carrier or vehicle. The compounds may be derivatized as the corresponding salts, esters, enol ethers or esters, acids, bases, solvates, hydrates or prodrugs prior to formulation, as described above. The concentrations of the compounds in the compositions are effective for delivery of an amount, upon administration, that reduces uric acid by at least 1 mg/dl. In a preferred embodiment, an amount of UALA agent is administered to lower serum uric acid below <5.5 mg/dl. In a more preferred embodiment, UALA is administered to produce serum uric acid levels between 4.0 mg/dl to 5.5 mg/dl. Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, particularly tumor-targeted liposomes, may also be suitable as pharmaceutically acceptable carriers. These may be prepared according to methods known to those skilled in the art. For example, liposome formulations may be prepared as described in U.S. Pat. No. 4,522,811.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo systems (see, e.g., Rosenthal et al. (1996) Antimicrob. Agents Chemother. 40(7):1600-1603; Dominguez et al. (1997) J. Med. Chem. 40:2726-2732; Clark et al. (1994) Molec. Biochem. Parasitol. 17:129; Ring et al. (1993) Proc. Natl. Acad. Sci. USA 90:3583-3587; Engel et al. (1998) J. Exp. Med. 188(4):725-734; Li et al. (1995) J. Med. Chem. 38:5031) and then extrapolated therefrom for dosages for humans.

According to another embodiment, the UALA is administered in conjunction with a diuretic agent. Acute renal failure is often treated with diuretics in an attempt to convert the patient from an oliguric to a nonoliguric state. Whereas this does result in better ease of management, there has been little evidence that diuretics actually accelerate recovery, despite the belief that they should since they would facilitate removal of casts etc from the renal tubules. The inventors have discovered that uric acid causes an acute fall in renal blood flow by micropuncture, which they have determined is due to the induction of endothelial dysfunction and impaired NO production. It is the inventors' belief that, since diuretics raise uric acid, and since uric acid is also elevated in subjects with acute renal failure, that diuretics raise uric acid causing a fall in renal blood flow that counters the beneficial effects of diuretics. Hence, one embodiment, in accordance with the inventors' realization that a diuretic alone may not effect recovery or prolong recovery, is an embodiment directed to a UALA administered in combination with a diuretic to thereby accelerate renal recovery. The UALA and diuretic may be administered together as components in an admixture or single composition, or in separate compositions administered at an appropriate time proximate to each other. Those skilled in the art will appreciate that known diuretics may be implemented in accord with this embodiment. Examples of common diuretics is found at http://lysine.pharm.utah.edu/netpharm/netpharm_{—}00/notes/diuretics.html.

It is known that tumor treatment in children can lead to exceptionally high serum uric acid levels, i.e., greater than 14 mg/dl. At such levels of serum uric acid, the uric acid in the blood forms crystals in the kidneys, which can lead to acute renal failure. This condition is known as acute urate nephropathy. This is one known cause of acute renal failure, however, there are many other circumstances in which acute renal failure arises, such as in a surgical setting as described above. Until the inventors' discoveries, the art has not been aware that acute renal failure may be brought about elevated serum uric acid levels not caused by tumor lysis, and at significantly lower serum uric acid levels exhibited upon tumor treatment. In many cases, acute renal failure occurs post-surgically and at levels well below 14 mg/dl. Thus, according to one embodiment, the subject invention pertains to a method of identifying whether a surgical patient is susceptible to post-operative acute renal failure. This method comprises determining a patient's serum uric acid level prior to surgery. In a specific embodiment, a surgical patient is determined to be susceptible to post-operative acute renal failure if the patient has a pre-operative or post-operative serum uric acid level of greater than 5.5 mg/dl. Further, in another embodiment a patient is determined to be susceptible to post-operative acute renal failure if the patient has a pre-operative and/or post-operative serum uric acid level between 6 mg/dl and 13 mg/dl.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to lower uric acid concentrations to levels <5.5 mg/dl in the serum.

Typically a therapeutically effective dosage should produce a serum concentration of active ingredient of from about 0.1 ng/ml to about 50-100 μg/ml. The pharmaceutical compositions typically should provide a dosage of from about 0.001 mg to about 2000 mg of compound per kilo-gram of body weight per day. Pharmaceutical dosage unit forms are prepared to provide from about 1 mg to about 1000 mg and preferably from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Preferred pharmaceutically acceptable derivatives include acids, bases, enol ethers and esters, salts, esters, hydrates, solvates and prodrug forms. The derivative is selected such that its pharmacokinetic properties are superior to the corresponding neutral compound.

Thus, effective concentrations or amounts of one or more of the compounds described herein or pharmaceutically acceptable derivatives thereof are mixed with a suitable pharmaceutical carrier or vehicle for systemic, topical or local administration to form pharmaceutical compositions. Compounds are included in an amount effective for reducing uric acid by at least 1 mg/dl with a target blood level of <5.5 mg/dl. The concentration of active compound in the composition will depend on absorption, inactivation, excretion rates of the active compound, the dosage schedule, amount administered, particular formulation as well as other factors known to those of skill in the art.

The compositions are intended to be administered by a suitable route, including orally, parenterally, rectally, topically and locally. For oral administration, capsules and tablets are presently preferred. The compositions are in liquid, semi-liquid or solid form and are formulated in a manner suitable for each route of adminimstration. Preferred modes of administration include parenteral and oral modes of administration. Oral administration is presently most preferred.

Solutions or suspensions used for parentetal, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount sufficient to alleviate the symptoms of the treated subject.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain 0.001%-100% active ingredient, preferably 0.1-85%, typically 75-95%.

The active compounds or pharmaceutically acceptable derivatives may be prepared with carriers that protect the compound against rapid elimination from the body, such as time release formulations or coatings.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, preferably capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included,

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

2. Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, preferably more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using knowvn testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound of formula I in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg, preferably 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, preferably 5-35 mg, more preferably about 9-30 mg of lyophilized powder, is added per mL of sterile water or other suitable carrier. The precise amount depends upon the selected compound. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5. Compositions for Other Routes of Administration

Other routes of administration, such as transdermal patches and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

6. Articles of Manufacture

The compounds or pharmaceutically acceptable derivatives may be packaged as articles of manufacture containing packaging material, a compound or pharmaceutically acceptable derivative thereof provided herein, which is effective for reducing serum uric levels.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,352. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for post-operative increases in serum uric acid.

### EXAMPLES

### Example 1

### Hyperuricemia Induces Arteriolopathy of Preglomerular Vessels that Impairs the Autoregulatory Response of Preglomerular Vessels Resulting in Glomerular Hypertension

### Methods

Experimental Design: All animal procedures were approved by the Animal Care Committee. Male 300-350 g Sprague-Dawley rats were used in all experiments.

Hyperuricemia in normal rats on normal sodium diet: To produce hyperuricemia, rats were administered oxonic acid (OA) (Sigma, St Louis Mo., USA) 750 mg/kg body weight by gastric gavage. To prevent the rise of serum uric acid induced by OA, allopurinol (AP) (Sigma, St Louis Mo., USA) was administered in drinking water (150 mg/L). We studied the following groups: Control (n=8); OA (n=7) and OA plus allopurinol (n=9). Systolic blood pressure and serum uric acid were measured at baseline and after 30-33 days of follow-up. Micropuncture studies were performed at 5 weeks.

Hyperuricemia in remnant kidney rats on normal sodium diet: Under light anesthesia with ether, 5/6 nephrectomy was performed by removal of the right kidney and selective ligation of 2-3 branches of left renal artery. To induce hyperuricemia rats were given OA (750 mg/kg) by gastric gavage starting the day after the renal ablation. To prevent hyperuricemia AP was administered in the drinking water (150 mg/L). The following groups were studied: 5/6 Nx (n=10); 5/6 Nx+OA (n=12) and 5/6 Nx+OA+AP (n=13). Systolic blood pressure, proteinuria and serum uric acid were measured at baseline and after 25-28 days of follow-up. Micropuncture studies were performed at 4 weeks.

Micropuncture: Animals were anesthetized with pentobarbital sodium (30 mg/kg ip) and placed on a thermoregulated table to maintain body temperature at 37° C. Trachea, jugular veins, femoral arteries, and the left ureter were catheterized with polyethylene tubing (PE-240, PE-50, and PE-10). The left kidney was exposed, placed in a Lucite holder, sealed with agar, and covered with Ringer's solution. Mean arterial pressure (MAP) was monitored with a pressure transducer (Model p23 db; Gould, San Juan, PR) and recorded on a polygraph (Grass Instruments, Quincy, Mass., USA). Blood samples were taken periodically and replaced with blood from a donor rat. Rats were maintained under euvolemic conditions by infusion of 10 ml/kg of body weight of isotonic rat plasma during surgery, followed by an infusion of 25% polyfructosan, at 2.2 ml/h (Inutest, Laevosan-Gesellschaft, Linz, Austria). After 60 min, five to six 3-min collection samples of proximal tubular fluid were obtained to determine flow rate and polyfructosan concentration. Intratubular pressure under free-flow and stop-flow conditions and peritubular capillary pressure were measured in other proximal tubules with a servo-null device (Servo Nulling Pressure System; Instrumentation for Physiology and Medicine, San Diego, Calif., USA). Polyfructosan was measured in plasma samples. Glomerular colloid osmotic pressure was estimated in protein from blood of the femoral artery (Ca) and surface efferent arterioles (Ce). Polyfructosan concentrations were determined by the technique of Davidson and Sackner (Davidson W D, Sackner Mass.: Simplification of the Antrhone Method for the Determination of Inulin in Clearance Studies. J. Lab Clin. Med. 62:351-356, 1963). The volume of fluid collected from individual proximal tubules was estimated from the length of the fluid column in a constant bore capillary tube of known internal diameter. Concentration of tubular polyfructosan was measured by the method of Vurek and Pegram (Vurek G G, Pegram S E: Fluorometric method for the determination of nanogram quantities of inulin. Ann. Biochem 16:409-419, 1966). Protein concentration in afferent and efferent samples was determined according to the method of Viets et al. (Viets J W, Deen W M, Troy J L, Brenner B M: Determination of serum protein concentration in nanoliter blood samples using fluorescamine or 9-phthalaldehyde. Anal. Biochem 88:513-521, 1978). MAP, GFR, single-nephron GFR (SNGFR), glomerular capillary hydrostatic pressure (Pgc), single-nephron plasma flow (QA), afferent (RA) and efferent (RE) resistances, and Kf, were calculated according to equations given elsewhere (Baylis C, Deen W M, Myers B D, Brenner B M: Effects of some vasodilator drugs on transcapillary fluid exchange in renal cortex. Am. J. Physiol 230:1148-1158, 1976).

Evaluation: In all studies, systolic blood pressure (SBP) was measured by tail cuff sphyngomanometer using an automated system (Narco Biosystems, Houston Tex., USA). All animals were preconditioned for blood pressure measurements 1 week before each experiment. Proteinuria was determined by turbidimetry by the method of trichloroacetic acid (Henry R J, Sobel Ch, Segalove M: Turbidimetric determination of proteins with sulfosalicylic and trichloroacetic acids. Proc Soc Exp Biol Med 92:748-751, 1956). Serum uric acid was measured by colorimetric phosphotungstic acid method using a commercial kit (Wiener Lab, Argentina).

Renal Histology: After the micropuncture study kidneys and remnant kidneys were washed by perfusion with PBS and then fixed with 4% paraformaldehyde. Renal biopsies were embedded in paraffin. Four μm sections of fixed tissue were stained with periodic acid Schiff (PAS) reagent. Arteriolar morphology was assessed by indirect peroxidase immunostaining for alpha-smooth muscle actin (DAIO Corp. Carpinteria, Calif., USA). In addition in remnant kidney rats glomerulosclerosis and fibrosis was assessed with Masson's trichrome stain and tubulointerstitial lymphocytes were assessed by indirect immunoperoxidase immunostaining for anti-CD-5.

Quantification of morphology: Quantifications were performed blinded. Arteriolopathy of afferent arterioles was evaluated as previously described (Sanchez-Lozada L G, Tapia E, Avila-Casado C, Soto V, Franco M, Santamaria J, Nakagawa T, Rodriguez-Iturbe B, Johnson R J, Herrera-Acosta J: Mild hyperuricemia induces glomerular hypertension in normal rats. Am. J. Physiol Renal Physiol 283:F1105-F1110, 2002). In brief, only vessels adjacent to glomeruli were selected. For each arteriole, the outline of the vessel and its internal lumen (excluding the endothelium) was generated using computer analysis to calculate the total medial area (outline-inline), in 10 arterioles per biopsy. The media/lumen ratio was calculated by the outline/inline relationship.

Glomerulosclerosis was evaluated in Masson's trichrome stained sections. The degree of sclerosis was scored as follows: in each biopsy the numbers of glomeruli with segmental, mesangial and global sclerosis as well as normal glomerular tufts were assessed. The resulting index in each animal was expressed as the percent of sclerosed glomeruli.

Tubulointerstitial fibrosis analysis was performed by a pathologist unaware of the origin of each kidney section. Evaluation was performed using slides stained with Masson's trichrome. Five noncrossed fields (770×58 mm, enlarged ×40) per biopsy were analyzed, using light microscopy (Olympus B-51. Olympus American, Melville, N.Y.) and captured with a digital video camera (CoolSnap Pro, Media Cybernetics Silver Spring, Md.). Each picture was processed on a computer and analyzed using Image-Pro and Photoshop 7 (Adobe Systems, SanJose, Calif.). Using the capabilities of color recognition by this software, a blue color was selected for positive areas. After selection, these areas were quantified (pixel unit) using the histogram function of the software. For each field, the number of positive areas was expressed as a fraction of the tubulointerstitial area (positive areas divided by the overall field area). Finally, for each biopsy, the mean fractional amount of positive areas was obtained by averaging the values obtained from 5 fields examined.

Tubulointerstitial lymphocyte infiltration was studied with the immunoperoxidase technique as described before (Rincon J, Parra G, Quiroz Y, Benatuil L, Rodriguez-Iturbe B: Cyclosporin A reduces expression of adhesion molecules in the kidney of rats with cluronic serum sickness. Clin. Exp Immunol. 121:391-398, 2000; Tapia E, Franco M, Sanchez-Lozada L G, Soto V, Avila-Casado C, Santamaria J, Quiroz Y, Rodriguez-Iturbe B, Herrera-Acosta J: Mycophenolate mofetil prevents arteriolopathy and renal injury in subtotal ablation despite persistent hypertension. Iidney Int. 63:994-1002, 2003). Briefly, tissues were successively washed and incubated with 20 ml of ExtrAvidin, 2.5 mg/ml (Sigma) and with 30 ml 0.001% biotin in phosphate buffered saline (PBS). Afterwards, tissues were incubated for 2 hours at 37° C. with 50 ml of the corresponding primary monoclonal antibody (see later) diluted 1:30 in Tris saline buffer (TSB), pH 7.8. After washing in TSB for 15 minutes, tissues were incubated for 1 hour with 30 ml of rat anti-mouse IgG (ab′) biotin-conjugated fragments with minimal cross reactivity with human, horse and rat serum proteins (Accurate Chemical Corp. Westbury, N.Y.), and finally for 30 minutes with 60 ml of peroxidase-conjugated ExtrAvidin. After a final wash, tissues were incubated for 15 minutes in diaminobenzidine and H₂O₂ in TSB.

The primary antibody used was anti CD5 (mouse monoclonal anti rat thymocytes and lymphocytes) (Biosource International, Camarillo, Calif.). Results were expressed as positive cells per mm².

Statistical analysis. Values are expressed as mean±standard error (SE). Differences between groups were evaluated by ANOVA with appropriate correction for multiple comparisons (Bonferroni). The relation between variables was assessed by correlation analysis.

### Results

### Hyperuricemia in Normal Rats on a Normal Sodium Diet:

General characteristics of the model: As previously described under low salt dietary conditions, oxonic acid administration induced a two-fold increment of serum uric acid in association with a slight, but significant, increment of systolic blood pressure demonstrated by intra-arterial and tail-cuff measurements (Sanchez-Lozada et al.) (Table 1). Concurrent administration of allopurinol prevented the increase of serum uric acid as well as the hypertension (Table 1). In addition individual values of serum uric acid and systolic blood pressure correlated positively (p<0.05). Values of body weight were similar among the studied groups (Table 1).

Micropuncture studies: Results of glomerular hemodynamic studies are summarized in Table 1. There were no changes in GFR among groups. Hyperuricemia was associated with cortical vasoconstriction as evidenced by a 35% decrease of SNGFR and negative correlation between individual values of uric acid and SNGFR (r=−0.6, p=0.004). The decrease in SNGFR resulted from lower QA and KF despite the counterbalancing effect of the significant increment of PGC. In addition, afferent and efferent resistances were 45% and 64% higher respectively. Concomitant treatment with allopurinol prevented these alterations, PGC and SNGFR remained unchanged and glomerular plasma flow increased to higher values than OA treated and control rats. Increment in QA was due to numerically lower values of AR and a significant decrease of ER. Additionally we found a negative correlation between serum uric acid and Kf (r=−0.5, p=0.02) and positive correlations of serum uric acid with PGC (r=0.7, p=0.0002), AR (r=0.5, p=0.007) and ER (r=0.7, p<0.0001).

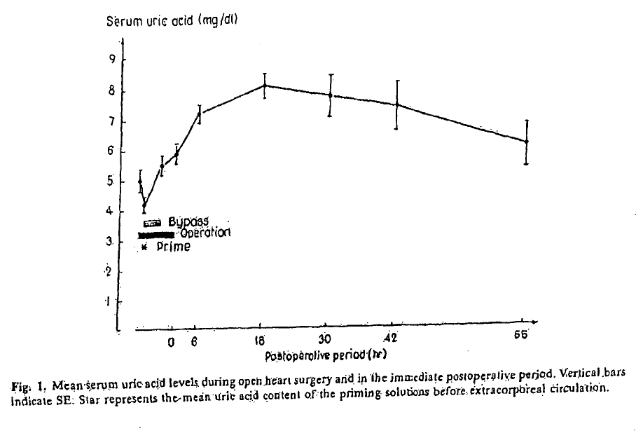

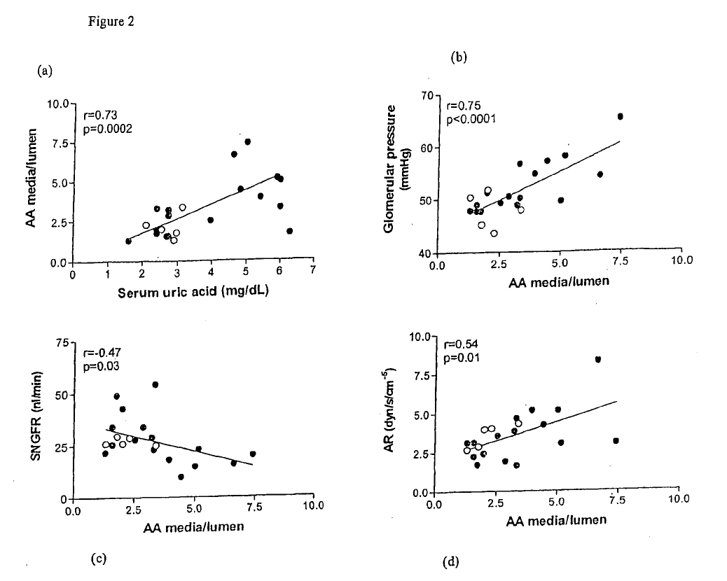

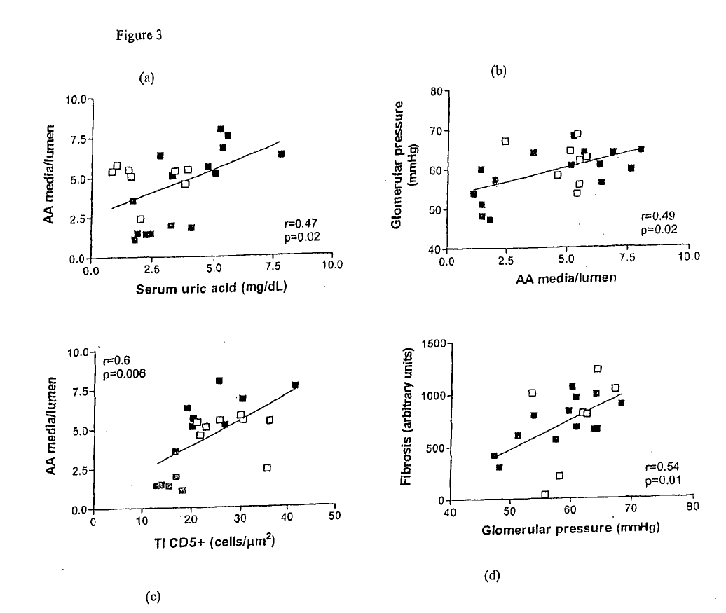

Histological examination: As previously reported in low salt dietary conditions, oxonic acid administration induced hypertrophy of afferent arteriole as disclosed by a significant increment of media to lumen ratio compared to control rats (Sanchez Lozada et al.) (Table 1). Allopurinol treatment prevented thickening of afferent arterioles (Table 1). We found positive correlations between individual values of serum uric acid and media to lumen ratio (r=0.73, p=0.0002); in addition, arteriolopathy (M/L) correlated positively with glomerular pressure (r=0.75, p<0.0001) and afferent resistance (r=0.54, p=0.01), finally a negative correlation between M/L and single nephron GFR was demonstrated (r=−0.47, p=0.03) (FIG. 1).

### Hyperuricemia in Remnant Kidney Rats.

General characteristics of the model: Renal ablation was characterized by marked hypertension, proteinuria and normal values of serum uric acid four weeks after surgery (Table 2). Administration of oxonic acid induced a two fold increase of serum uric acid (p<0.001) that was associated with further increase of 30 mmHg of SBP (p<0.05) and a two fold additional rise of proteinuria (p<0.01) (Table 2). Concomitant administration of allopurinol prevented hyperuricemia (p<0.001) and proteinuria (p<0.01) but had only a marginal effect on SBP (p=ns) (Table 2). There were no differences of body weight among studied groups (Table 2).

Micropuncture studies: The results obtained in the micropuncture studies are summarized in Table 2. Although whole kidney GFR was similarly reduced by renal ablation in all groups (table 2), measurement of single nephron function demonstrated marked differences in glomerular adaptation between RK control rats and those receiving OA and OA+AP. RK control rats showed the typical hyperfiltration as has been reported in other studies. In contrast, SNGFR in OA treated was 40% lower than the RK control group (40.6 vs 65 nl/min, p<0.01). Treatment with AP partially prevented the change of SNGFR (53 nl/min, p=ns).

The reduction of SNGFR in OA treated animals resulted from reduction of two of the determinants of GFR, the glomerular plasma flow and the ultrafiltration coefficient Kf, while glomerular capillary pressure persisted with similar elevations as the RK control group (62 vs 61 mmHg, p=ns). In the OA group glomerular plasma flow was 40% and Kf 50% lower than RK control group (GPF: 147 vs 243 nl/min, p<0.01. Kf: 0.02 vs 0.04 nl/s/mmHg, p<0.05). Glomerular under-perfusion in hyperuricemic rats was the result of a 120% higher afferent resistance and 80% higher efferent resistance compared to RK (control animals (AR: 4.6 vs 2.1 dyn/s/cm⁻⁵, p<0.01. ER: 1.8 vs 1 dyn/s/cm⁻⁵, p<0.01). Thus, despite vasoconstriction induced by hyperuricemia glomerular hypertension persisted unchanged. Treatment with AP partially prevented the cortical ischemia (Table 2), QA and SNGFR were numerically higher than hyperuricemic rats. However the most striking effect of AP was the prevention of glomerular hypertension in the presence of systemic hypertension. In fact PGC was normal and significantly lower than RK hyperuricemic rats (p<0.001) as well as lower than RK control (p<0.01), indicating that allopurinol treatment restored autoregulatory capacity of preglomerular arteriole to prevent increased transmission of pressure to the glomerular capillary.

Histological examination: Results of histological examination are summarized in table 3. Analysis of afferent arteriole morphology by immunostaining for alpha-smooth muscle actin demonstrated that extensive renal mass reduction induced thickening of the afferent arteriole wall evaluated by the media to lumen ratio, as we previously reported (Tapia et al.). Oxonic acid administration considerably aggravated hypertrophy of preglomerular vessels as indicated by higher media to lumen ratio than control (6.4 vs 4.9, p<0.05). Allopurinol prevented arteriolopathy in hyperuricemic to a greater extent than that observed in RK control rats (AP=1.8, p vs Nx<0.001, p vs OA<0.001). Moreover media to lumen values in OA+AP were similar to values found in the present study in normal rats (Table 1) using the same method of perfusion and fixation. This suggests that allopurinol exerted an additional favorable effect besides preventing hyperuricemia.

Neither glomerulosclerosis nor tubulointerstitial fibrosis were different among studied groups. Nevertheless tubulointerstitial infiltration of CD5 positive cells was significantly lower in allopurinol treated rats with respect to RK control and RK hyperuricemic rats (Nx=27.8; OA=26.1; OA+AP=15.8. Nx vs OA+AP p<0.01; OA vs OA+AP p<0.05).

Finally we found positive correlations between individual values of serum uric acid versus media to lumen ratio (r=0.5, p=0.02), M/L vs PGC (r=0.5, p=0.02) and CD5 (r=0.6, p=0.006), and PGC versus fibrosis (r=0.5, p=0.02) and CD5 (r=0.5, p=0.01).

### Discussion

The effect of hyperuricemia-induced preglomerular arteriolopathy on glomerular hemodynamics in normal and in remnant kidney rats on normal salt diet was studied. The main finding was that the vascular lesion was associated with cortical vasoconstriction and glomerular hypertension in both conditions. Previously the inventors studied the effect of mild hyperuricemia in normal rats under low salt diet and obtained similar results (Sanchez Lozada et al.), but the vasoconstrictive effect suggested in clinical could not be clearly demonstrated (Messerli F H, Frohlich E D, Dreslinski G R, Suarez D H, Aristimuno G G: Serum uric acid in essential hypertension: an indicator of renal vascular involvement. Ann. Intern. Med. 93:817-821, 1980) and experimental (Sesoko S, Pegram B L, Willis G W, Frohlich E D: DOCA-salt induced malignant hypertension in spontaneously hypertensive rats. J. Hypertens. 2:49-54, 1984) studies. In addition to differences in salt intake, other differences between both studies should be mentioned. In order to give rats more accurate doses of OA and achieve better inhibition of uricase, in this work oxonic was administered by daily gavage instead of a rat chow supplemented with 2% OA. Control values of SUA were higher in the present study than values reported in the previous studies under conditions of salt restriction (Sanchez Lozada et al.). Despite these differences, oxonic acid administration did significantly increase serum uric acid and allopurinol prevented this increment. Moreover since all animals were maintained in the same conditions comparisons remain valid.

In normal rats, eliminating RAS activation induced by low salt diet we were able to reveal the renal vasoconstrictive effect of hyperuricemia. In fact, SNGFR was 35% lower than controls and we found a negative correlation between individual values of SUA and SNGFR and KF as well as a positive correlation between afferent and efferent resistances. These findings are in agreement with previous experimental and clinical studies in which, a positive correlation between SUA and renal vascular resistance was observed.

In this study, it was confirmed that hyperuricemia produced arteriolopathy of preglomerular vessels as indicated by the positive correlation between individual values of serum uric acid and media to lumen ratio. Furthermore rats on normal salt intake had 10 mmHg less SBP than animals on LSD, and still had similar values of media to lumen ratio. There are evidences that uric acid directly stimulates vascular smooth muscle cell proliferation which may be the mechanism for the development of the vascular disease. Rao et al reported that uric acid stimulates PDGF-A chain expression and mediates cell proliferation in cultured vascular smooth muscle cells (Rincon J, Parra G, Quiroz Y, Benatuil L, Rodriguez-Iturbe B: Cyclosporin A reduces expression of adhesion molecules in the kidney of rats with chronic serum sickness. Clin. Exp Immunol. 121:391-398, 2000). Consistent with this report Kang et al found de novo expression of COX-2 mRNA and proliferation after incubation of VSMC with uric acid. A COX-2 inhibitor or a TXA2 receptor inhibitor prevented the proliferation in response to uric acid. In vivo COX-2 was also shown to be expressed de novo in the preglomerular vessels and its expression correlated both with the uric acid levels and with the degree of smooth muscle cell proliferation (Kang D H, Nakagawa T, Feng L, Watanabe S, Han L, Mazzali M, Truong L, Harris R, Johnson R J: A role for uric acid in the progression of renal disease. J. Am. Soc. Nephrol. 13:2888-2897, 2002). The present findings agree with these studies which demonstrated that Uric acid induces arteriolar hypertrophy by a mechanism independent of the increment of blood pressure. Moreover, in the present study the deleterious effect of arteriolopathy on glomerular hemodynamics was evidenced by the positive correlations between media to lumen ratio and glomerular pressure and afferent resistance and negative correlation between M/L versus SNGFR.

In summary these results suggest that in normal rats a mild increment of serum uric acid induces preglomerular arteriolopathy that results in transmission of systemic hypertension to glomerular capillary tuft which, in concert with higher efferent resistance induced by hyperuricemia, further enhance glomerular hypertension. In addition thickening of the afferent arteriole wall induces higher afferent resistance, and the reduction of glomerular plasma flow and Kf result in lower single nephron GFR. Systemic hypertension could result from the consequence of the decrement in filtered load or as a consequence of renal ischemia resulting from the decrease in renal blood flow.

Previous studies by Kang et al showed that hyperuricemia can accelerate progression of renal damage (Kang et al.). In that study, in rats with subtotal renal ablation induced by polectomy, mild increment of serum uric acid was associated with worse renal function, greater structural damage and severe vascular damage. Since we have shown that arteriolopathy of afferent arteriole in normal (Sanchez Lozada et al.) and 5/6 nephrectomy rats (Rincon et al.) is associated with alterations in glomerular microcirculation that contribute to perpetuate progression of renal damage, in the present study we evaluated glomerular hemodynamics changes caused by the vascular lesion induced by hyperuricemia in rats with 5/6 neplirectomy produced by ligation of several branches of the renal artery. Control 5/6 Nx rats developed severe hypertension and proteinuria, as has been reported in this model of renal ablation (Rincon et al.). Both parameters increased further with hyperuricemia, allopurinol restored proteinuria and tended but did not significantly reduce the blood pressure. The main finding of these studies was that in remnant kidney rats, mild hyperuricemia produced profound renal cortical vasoconstriction. Single nephron glomerular plasma flow and GFR fell by 40%, ultrafiltration coefficient Kf also decreased in the same proportion. Afferent and efferent resistances increased by two fold. Despite intense vasoconstriction, glomerular hypertension was unchanged in hyperuricemic rats. As we showed previously, morphometric analysis of afferent arteriole in control RK rats displayed hypertrophy of the vascular wall (Rincon et al.). However the rise in serum uric acid markedly accentuated the arteriolopathy by thickening the arteriolar wall and increasing the media to lumen ratio by 30%. Allopurinol treatment partly restored the functional changes, however it fully prevented arteriolopathy. Arteriolar wall thickness was normal and media to lumen ratio significantly lower than hyperuricemic and control RK groups. Interestingly, preservation of vascular structure was associated with maintaining normal glomerular pressure, despite systemic hypertension, which denotes a normal autoregulatory response of preglomerular vessels to the rise of arterial pressure. The contribution of preglomerular vascular lesion in determining the elevation of glomerular capillary pressure was further evidenced by a positive significant correlation of individual values of media to lumen ratio and glomerular pressure. In addition, a significant positive correlation was found between individual values of serum uric acid and media to lumen ratio underscoring the role of uric acid in stimulating proliferation of vascular smooth muscle cells.

Recently, it was reported that arteriolopathy of afferent arteriole by perpetuating glomerular hemodynamic stress contributes to progression of renal disease (Rincon et al.). In renal ablated rats histological examination and morphometry showed thickening of afferent arterioles as disclosed by a significant increase in media/lumen ratio indicating hypertrophy of the vessel wall. Proliferation of vascular smooth muscle cells and increased collagen deposition might be expected to increase rigidity of the vascular wall and thus limit its capacity to contract in response to higher perfusion pressure (Touyz R M, He G, El Mabrouk M, Schiffrin E L: p38 Map kinase regulates vascular smooth muscle cell collagen synthesis by angiotensin II in SHR but not in WKY. Hypertension 37:574-580, 2001). Maneuvers that prevent the accompanying inflammatory process, like treatment with the heparinoid pentosan polysulfate (Bobadilla N A, Tack I, Tapia E, Sanchez-Lozada L G, Santamaria J, Jimenez F, Striker L J, Striker G E, Herrera-Acosta J: Pentosan polysulfate prevents glomerular hypertension and structural injury despite persisting hypertension in 5/6 nephrectomy rats. J. Am. Soc. Nephrol. 12:2080-2087, 2001) and the immunosuppressive drug, mofetil mycophenolate (Rincon et al.) preserved arteriolar structure maintaining normal glomerular pressure despite persisting systemic hypertension indicating a normal autoregulatory response. In the present study, allopurinol treatment reduced tubulointerstitial infiltration of CD5+ cells compared to OA treated and RK control rats. This effect could be partially responsible of the fully prevention of preglomerular arteriolopathy and glomerular hypertension in allopurinol treated group besides its lowering uric acid effect.

On the other hand, hypertrophy of vascular smooth muscle cells and expanded ECM on vascular wall may critically reduce the lumen of preglomerular vessels inducing the decline of blood flow to glomeruli and post-glomerular ischemia which is a well-known stimulus to produce tubulointerstitial fibrosis and salt-sensitive hypertension (Johnson R J, Herrera-Acosta J, Schreiner G F, Rodriguez-Iturbe B: Subtle acquired renal injury as a mechanism of salt-sensitive hypertension. N. Engl. J. Med. 346:913-923, 2002). In the present study glomeruloesclerosis and fibrosis were similar among studied groups. However the correlation between individual values of glomerular pressure and fibrosis suggests that transmission of systemic hypertension to peritubular capillaries may damage them, further incrementing ischemia and fibrosis. In addition to these hemodynamic effects, experimental hyperuricemia has also been associated with endothelial dysfunction and lower serum levels of nitrates/nitrites indicating decreased synthesis of nitric oxide (Finch J, Mu W, Parra G, Feig D, Price K, Long D, Kang D H, Prabhakar S, Johnson R J: Hyperuricemia induces endothelial dysfunction in rats. (abstract) J. Am. Soc Nephrol. 14:143 A, 2004). In this regard, it was shown that inhibition of NO production markedly accelerates progression of renal damage independently of systemic hypertension and in association with loss of microvascular endothelium (Kang D H, Nakagawa T, Feng L, Johnson R J: Nitric oxide modulates vascular disease in the remnant kidney model. Am. J. Pathol. 161:239-248, 2002). Bohle et al also suggested a critical role for the peritubular capillaries in the progressive interstitial fibrosis in human renal disease (Bohle A, Mackensen-Haen S, Wehrmann M: Significance of postglomerular capillaries in the pathogenesis of chronic renal failure. Kidney Blood Press Res. 19:191-195, 1996).

In conclusion renal arteriolopathy induced by mild hyperuricemia results in an impaired capacity of preglomerular vessels to maintain constancy of glomerular pressure in the phase of arterial hypertension which results in glomerular hypertension. In addition, lumen obliteration induced by vascular wall thickening results in severe vasoconstriction decreasing renal plasma flow, GFR and perfusion to peritubular capillaries. The resulting ischemia is a potent stimulus that induces tubulointerstitial inflammation and fibrosis as well as arterial hypertension. Evidence for these alterations is provided by studies in which mild hyperuricemia was responsible for later development of salt sensitive hypertension in normal rats (Watanabe S, Kang D H, Feng L, Nakagawa T, Kanellis J, Lan H, Mazzali M, Johnson R J: Uric acid, hominoid evolution, and the pathogenesis of salt-sensitivity. Hypertension 40:355-360, 2002). Furthermore, it is possible that as the arteriolar changes become even more severe glomerular ischemia and collapse with a decrease in glomerular pressure could take place, as has been postulated to occur with prolonged and severe hypertension (Ratschek M, Ratschek E, Bohle A: Decompensated benign nephrosclerosis and secondary malignant nephrosclerosis. Clin. Nephrol. 25:221-226, 1986). Thus, these studies provide a mechanism by which hyperuricemia can mediate hypertension and renal disease.

TABLE 1
Glomerular hemodynamics and afferent arteriole morphology in normal rats on normal salt diet.

| | SUA | SBP | BW | MAP | GFR | PGC | SNGFR |
|---|---|---|---|---|---|---|---|
| Groups | mg/dL | mmHg | gr | mmHg | ml/min | mmHg | nl/min |
| Normal | 2.5 ± 0.2 | 124 ± 0.8 | 326 ± 14 | 113 ± 4 | 0.81 ± 0.1 | 47 ± 1 | 27.6 ± 0.8 |
| OA | 5.4 ± 0.2 | 134 ± 3 | 341 ± 16 | 131 ± 4 | 0.71 ± 0.1 | 56 ± 2 | 17.8 ± 2 |
| OA + AP | 2.6 ± 0.2 | 123 ± 2 | 345 ± 4 | 124 ± 4 | 0.87 ± 0.1 | 49 ± 0.4 | 35.4 ± 4 |
| N vs OA | <0.001 | <0.01 | ns | <0.05 | ns | <0.001 | <0.05 |
| OA vs OA + AP | <0.001 | <0.01 | ns | ns | ns | <0.001 | <0.001 |
| N vs OA + AP | ns | ns | ns | ns | ns | ns | ns |

| | QA | RA | RE | KF | |
|---|---|---|---|---|---|
| Groups | nl/min | dyn/s/cm⁻⁵ | dyn/s/cm⁻⁵ | nl/s/mmHg | AA M/L |
| Normal | 76.2 ± 3.3 | 3.33 ± 0.22 | 1.73 ± 0.10 | 0.035 ± 0.004 | 2.2 ± 0.3 |
| OA | 66.1 ± 7.2 | 4.82 ± 0.67 | 2.83 ± 0.19 | 0.027 ± 0.002 | 5.1 ± 0.6 |
| OA + AP | 127.4 ± 15.5 | 2.64 ± 0.27 | 1.21 ± 0.13 | 0.036 ± 0.003 | 2.2 ± 0.3 |
| N vs OA | ns | ns | <0.001 | ns | <0.001 |
| OA vs OA + AP | <0.01 | <0.01 | <0.001 | ns | <0.001 |
| N vs OA + AP | <0.01 | ns | <0.05 | ns | ns |

TABLE 2
Glomerular hemodynamics in remnant kidney rats.

| | SUA | SBP | Uprot | BW | MAP | GFR | PGC |
|---|---|---|---|---|---|---|---|
| Groups | mg/dL | mmHg | mg/d | gr | mmHg | ml/min | mmHg |
| RK | 2.1 ± 0.4 | 161 ± 6 | 127 ± 30 | 352 ± 11 | 163 ± 7 | 0.4 ± 0.1 | 61 ± 2 |
| OA | 4.5 ± 0.4 | 191 ± 10 | 246 ± 29 | 330 ± 15 | 184 ± 6 | 0.4 ± 0.1 | 62 ± 1 |
| OA + AP | 2.5 ± 0.2 | 184 ± 5 | 112 ± 17 | 307 ± 12 | 170 ± 6 | 0.4 ± 0.04 | 53 ± 2 |
| RK vs OA | <0.001 | <0.05 | <0.01 | ns | ns | ns | ns |
| OA vs OA + AP | <0.001 | ns | <0.01 | ns | ns | ns | <0.001 |
| RK vs OA + AP | ns | ns | ns | ns | ns | ns | <0.01 |

| | SNGFR | QA | RA | RE | KF |
|---|---|---|---|---|---|
| Groups | nl/min | nl/min | dyn/s/cm⁻⁵ | dyn/s/cm⁻⁵ | nl/s/mmHg |
| RK | 65.3 ± 4.3 | 242.5 ± 24.7 | 2.11 ± 0.25 | 1.02 ± 0.11 | 0.041 ± 0.004 |
| OA | 40.7 ± 6.0 | 146.5 ± 20.4 | 4.55 ± 0.66 | 1.83 ± 022 | 0.024 ± 0.003 |
| OA + AP | 52.7 ± 4.0 | 172.6 ± 17.8 | 3.52 ± 0.39 | 1.28 ± 0.11 | 0.044 ± 0.005 |
| RK vs OA | <0.01 | <0.01 | <0.01 | <0.01 | <0.05 |
| OA vs OA + AP | ns | ns | ns | ns | <0.01 |
| RK vs OA + AP | ns | ns | ns | ns | ns |

TABLE 3
Histological findings in remnant kidney rats.

| | | | Glomerulo- | |
|---|---|---|---|---|
| | | TI Fibrosis | sclerosis | TI CD5 + |
| Groups | AA M/L | arbitrary units | % | cells/mm² |
| RK | 5.0 ± 0.39 | 733.3 ± 156.3 | 0.24 ± 0.10 | 27.8 ± 2.13 |
| OA | 6.4 ± 0.37 | 780.2 ± 55.0 | 0.33 ± 0.07 | 26.1 ± 2.99 |
| OA + AP | 1.8 ± 0.31 | 676.9 ± 107.3 | 0.27 ± 0.04 | 15.8 ± 0.80 |
| RK vs OA | <0.05 | ns | ns | ns |
| OA vs OA + | <0.001 | ns | ns | <0.05 |
| AP | | | | |
| RK vs OA + | <0.001 | ns | ns | <0.01 |
| AP | | | | |

### Example 2

### Hyperuricemia Induces Endothelial Dysfunction by Inhibiting the Production of NO in Rats

### Methods

Male Sprague-Dawley rats were housed in standard conditions and fed normal diets. We induced hyperuricemia with an uricase inhibitor, oxonic acid (OA; 750 mg/kg/day), by gavage, with control rats receiving vehicle. Allopurinol (AP) was used to block hyperuricemia by placing AP in the drinking water (150 mg/L). Rats were divided into four groups: (1) Control, (2) AP only, (3) OA only, and (4) OA+AP. Systolic blood pressure was measured using a tail-cuff sphygmomanometer. The amount of drinking water consumed and changes in body weight were noted. Rats were sacrificed at one and seven days. Serum was analyzed for uric acid concentration and nitrites/nitrates (NOₓ) by chemiluminescence method. (Prabhakar SS: Inhibition of mesangial iNOS by reduced extracellular pH is associated with uncoupling of NADPH oxidation. Kidney Int 61:2015-2024, 2002). Statistical analysis between subgroups was performed using ANOVA.

### Results

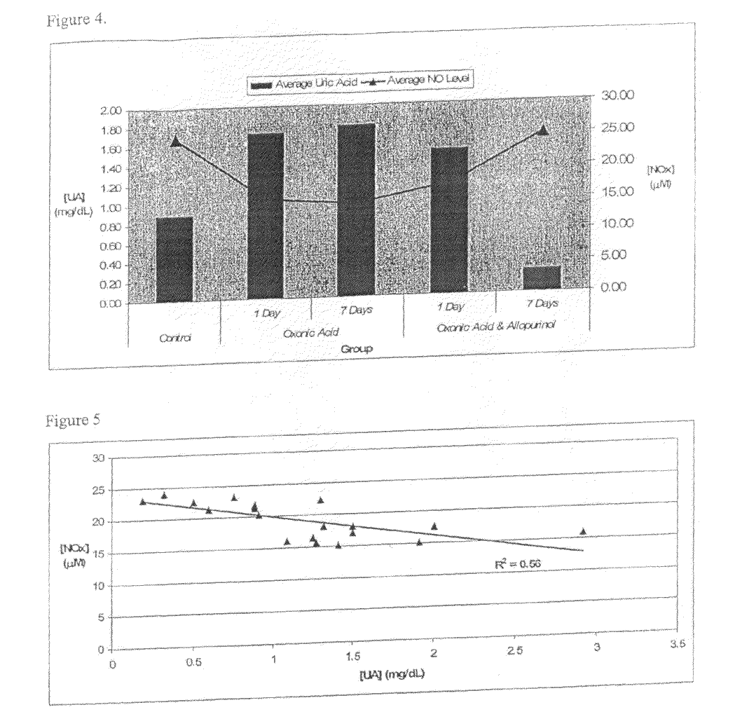

There was no difference in the amount of water consumed and the change in body weight between the three groups over seven days. OA induced a mild hyperuricemia at both 1 day (1.7±0.7 vs. 0.8±0.4 mg/dL in OA vs. Control, p<0.05) and 7 days (1.8±0.4 vs. 0.9±0.7 mg/dL in OA vs. Control, p<0.05). AP only had a mild and non-significant effect on serum uric acid concentrations at day 1 (1.52±0.3 mg/dL, p=NS), but effectively reversed the hyperuricemia at 7 days (0.3±0.2 mg/dL, p<0.001). Serum nitrites and nitrates (NOₓ) were reduced by 40-50% in hyperuricemic rats at both 1 day (15.6±0.4 vs. 22.6±1.0 μmol/L in OA vs. Control, p<0.001) and 7 days (14.6±1.1 vs. 27.5±1.3 μmol/L in OA vs. Control, p<0.001). This decrease in NOₓ was improved slightly by AP at 1 day (17.4±0.8 μmol/L, p<0.001) and reversed completely at 7 days (25.0±0.8 μmol/L, p<0.001). (FIG. 4.) There was also a direct linear correlation between serum UA and NOₓ (FIG. 5). Rats treated with AP alone did not show a significant change in either serum UA or NOₓ concentration. Rats treated with OA also showed a trend toward higher systolic blood pressure at 7 days (178±18 vs. 158±16 vs. 147±11 mm Hg in OA vs. Control vs. OA/AP, p=NS).

### CONCLUSIONS

Most mammals have the enzyme uricase that degrades uric acid to allantoin with the generation of oxidants. In humans, uricase is mutated resulting in higher uric acid levels. Rats administered an uricase inhibitor (oxonic acid) develop mild hyperuricemia, hypertension, and vascular disease that is mediated by activation of the renin-angiotensin system, a loss of macula densa NO synthase, and the development of microvascular disease (Mazzali M, Hughes J, Kim Y G, Jefferson J A, Kang D H, Gordon K L, Lan H Y, Kivlighn S, Johnson R J: Elevated uric acid increases blood pressure in the rat by a novel crystal-independent mechanism. Hypertension 38:1101-1106, 2001). In this study we also demonstrate that hyperuricemic rats have a fall in serum nitrites (a reflection of NO production) that is reversed by allopurinol. Furthermore, there was a direct linear correlation between serum uric acid and serum nitric oxide. The induction of hyperuricemia also showed a trend towards increased systolic blood pressure. This data shows that hyperuricemia leads to endothelial dysfunction in the rat. Interestingly, Waring et al recently reported that the infusion of uric acid into humans does not impair endothelial function over a one hour period (Waring W S, Adwani S H, Breukels O, Webb D J, Maxwell S R: Hyperuricaemia does not impair cardiovascular function in healthy adults. Heart 90:155-159, 2004). However, these studies did not measure nitric oxide levels nor mention effects of sustained hyperuricemia on endothelial-dependent vasodilatation. These discrepancies can also be explained by differences in methods and species, suggesting the need for further studies to dissect out the complex relationship of uric acid to endothelial function and cardiovascular disease.

What is claimed is:

1. A method of preventing or accelerating recovery of post-operative ARF comprising:
identifying whether a patient of an operative procedure is at risk of experiencing post-operative ARF; and
administering to said patient a composition comprising an effective amount of UALA to lower serum uric acid to below 5.5 mg/dl.

2. The method of claim 1, wherein said administering occurs seven days or less prior to said operative procedure.

3. The method of claim 2, wherein said administering occurs 3 days or less prior to said operative procedure.

4. The method of claim 1, wherein said administering occurs during said operative procedure.

5. The method of claim 1, wherein said administering occurs 24 hours or less following said operative procedure.

6. The method of claim 1, wherein said administering occurs 12 hours or less following said operative procedure.

7. The method of claim 1, wherein said administering occurs 8 hours or less following said operative procedure, and wherein said administering comprises parenteral administration of said composition.

8. The method of claim 1, wherein said administering occurs 4 hours or less following said operative procedure.

9. The method of claim 1, wherein said administering occurs 2 hours or less following said operative procedure.

10. The method of claim 1, wherein said UALA is rasburicase.

11. The method of claim 1, further comprising
obtaining a serum sample from said patient post-operatively; and
determining uric acid concentration in said serum sample.

12. A method of preventing or accelerating recovery of ARF comprising:
determining renal function of a patient;
determining uric acid concentration in a serum sample obtained from said patient;
identifying whether a patient is at risk of experiencing post-operative ARF; and
administering to said patient a composition comprising UALA.

13. A method of preventing or decelerating progression of uric acid induced renal failure comprising administering an effective amount of UALA to a patient susceptible to uric acid induced renal failure.

14. The method of claim 13, wherein said UALA is administered over the course of at least one week.

15. The method of claim 13, wherein said UALA is administered over the course of at least 2 weeks.

16. The method of claim 13, wherein said UALA is administered over the course of at least 4 weeks.

17. The method of claim 13, wherein said UALA is administered in a regimen to maintain the average serum uric acid levels equal to or below 5.5 mg/dl for at least 2 weeks.

18. The method of claim 17, wherein said regimen maintains average serum uric acid levels equal to or below 5.5 mg/dl for at least 4 weeks.

19. The method of claim 17, wherein said regimen maintains average serum uric acid levels equal to or below 5.5 mg/dl for at least 8 weeks.

20. The method of claim 17, wherein said regimen maintains average serum uric acid levels equal to or below 5.5 mg/dl for at least 24 weeks.

21. The method of claim 13, wherein said UALA is administered in an amount to produce a serum uric acid level of 4.0 mg/dl to 5.5 mg/dl for a sufficient period of time to prevent or decelerate progression of uric acid induced renal failure.

22. The method of claim 13, wherein said patient is a cardiovascular surgery patient.

23. The method of claim 13, wherein said patient is a chronic hyperuricemic patient.

24. A method of preventing, diminishing or accelerating recovery from uric acid induced renal failure comprising administering an effective amount of UALA and a diuretic to a patient susceptible to or suffering from uric acid induced renal failure.

25. The method of claim 24, wherein said administering produces a serum uric acid level of 4.0 mg/dl to 5.5 mg/dl for a sufficient period of time to prevent, decelerate progression of, or accelerate recovery from uric acid induced renal failure.
